(19) United States
(12) Patent Application Publication
(10) Pub. No.: US 2023/0089466 A1
(43) Pub. Date: Mar. 23, 2023

(54) Establishment of Identification and Screening Method of Cows with A2 Beta-Casein Genotype of Producing A2 Milk and Applications Thereof

(71) Applicant: Huazhong Agricultural University, Wuhan (CN)

(72) Inventors: Shujun Zhang, Wuhan (CN); Haitong Wang, Wuhan (CN); Liangkang Nan, Wuhan (CN); Yikai Fan, Wuhan (CN); Xuelu Luo, Wuhan (CN)

(21) Appl. No.: 17/846,008

(22) Filed: Jun. 22, 2022

(30) Foreign Application Priority Data
Sep. 10, 2021 (CN) 202111063302X

(51) Int. Cl. G01N 21/35; G06K 9/62; G01N 33/04

(52) U.S. Cl. CPC G01N 21/35; G06K 9/6256; G06K 9/6269; G06K 9/6282; G01N 33/04

(57) ABSTRACT

A fast, batch and non-invasive identification and screening method of cows with A2 β-casein genotype of producing A2 milk belongs to the cow genotyping field and includes steps of: 1) collecting milk samples of cows; 2) detecting and acquiring mid-infrared spectral data; 3) data preprocessing to remove outliers; 4) dividing the dataset after the preprocessing into a training set and a testing set according to random sampling principle; 5) screening modeling spectral wavebands; and 6) combining different spectrum preprocessing methods with a modeling algorithm to establish classification models, using accuracy, sensitivity, specificity and AUC to evaluate the models, and determining the best classification model. The method can realize rapid and batch identification of cows of producing A2 milk and non A2 milk, and may have advantages of fast, high precision, low cost, simple operation, batch determination, no trauma (no blood collection, hair plucking, and tissue extraction), and strong practicability.

### TECHNICAL FIELD

The invention belongs to the field of cow genotyping technologies, and in particularly to an establishment of fast, batch and non-invasive identification and screening method of cows with A2 β-casein genotype of producing A2 milk and an application thereof.

### BACKGROUND

Milk is a natural high quality food rich in nutrients and functional substances, and proteins in the milk are an important source of protein nutrition for human beings. The largest amount of milk protein (accounts for 70-80%) is caseins, of which β-casein accounts for about 30% of four types of caseins in the milk. Studies have shown that during the long-term evolution and improved breeding of dairy cows, mutations were found in the β-casein genes of dairy cows, and there are 13 kinds of genotypes; A1 genotype (referred to as A1 type) and A2 genotype (referred to as A2 type) have high percentages in the dairy herd, and the genetic codon (three bases) encoding an amino acid at the 67^{th} position of A2 protein has mutated from proline to histidine and thereby mutated to the A1 type cow. According to research reports, the A1 genotype β-casein milk (shorted as A1 milk) produced by the A1 type cow may increase the risk of type I diabetes in infants and may also cause immune response and functional impairment of digestion and respiration, and drinking the A1 milk may increase the risk of cardiovascular diseases such as heart disease and atherosclerosis, and type I diabetes. The A2 genotype β-casein milk (shorted as A2 milk) produced by the A2 type cow can eliminate or alleviate the above symptoms. Accordingly, the A2 milk has better nutritional and functional values and thus has higher price and better market potential. Therefore, it is necessary to identify and screen a large number of A2 homozygous genotype cows with β-casein of producing A2 milk.

An existing screening method of A2 homozygous genotype cows with β-casein is to collect hair or blood from cows and perform genotyping detection through DNA analysis. This method can accurately classify β-casein genotypes carried by the cows, but its cost is high (about RMB 50 yuan per cow, or more) and the blood or hair collection process would cause stress and trauma to the cow. At present, immunology, gel electrophoresis, liquid chromatography-mass spectrometry, liquid chromatography, reverse-phase high performance liquid chromatography (RP)-high performance liquid chromatography (HPLC), capillary electrophoresis (CE), and urea polyacrylamide electrophoresis are used to identify or quantitatively analyze A1 and A2 β-caseins in dairy products based on protein levels; although accurate results can be achieved, the above methods have high requirements on technology, time, cost, sample size, instrument and operator skill, and thus cannot meet the requirements of rapid and mass inspection in this field.

The mid-infrared spectroscopy is a fast, non-destructive, non-invasive, nuisanceless and multi-component simultaneous analysis modern technology which has been developed rapidly in recent years. Machine learning algorithms used to establish classification models include decision tree, naive Bayes, artificial neural network, bootstrap aggregation, K-nearest neighbor, random forest (RF), and support vector machine. In practice, the random forest and the support vector machine have better performance with low error rate, high accuracy, high sensitivity and high specificity. Data output by a mid-infrared spectrometer is a matrix of n×1060 (n is the sample size), the data are huge, and it is difficult to avoid incomplete and inconsistent data, and is highly susceptible to noise (errors or outliers). Low quality data would lead to poor data mining results, and therefore it is necessary to study appropriate methods to effectively process the output data. These methods usually include data normalization, processing missing values, removing noise and outliers, and feature selection. For example, using first-order differentiation, standard normal variate transform (SNV), multiplicative scatter correction (MSC) and Savitzky-Golay (SG) convolution smoothing to mine differences of classification objects, and using the Mahalanobis distance to remove outliers. However, it is still not clear which method is suitable for the detection of A2 β-casein genotype cows.

### SUMMARY

In order to solve the above-mentioned problems, an embodiment of the invention provides a fast, batch and non-invasive identification and screening method of cows with A2 β-casein genotype of producing A2 milk.

To achieve the above purpose, in an aspect, a technical solution proposed by the invention may be as follows.

A fast, batch and non-invasive identification and screening method of cows with A2 β-casein genotype of producing A2 milk, may include following steps:

1) collecting milk samples, including:

collecting raw milk from a dairy farm of breeding cows with A2 β-casein genotype of producing A2 milk and cows with non-A2 genotype of producing non-A2 milk, to obtain A2 β-casein milk (A2 milk) samples and non-A2 β-casein milk (non-A2 milk) samples;

2) acquiring mid-infrared spectra, including:

scanning collected milk samples by using a milk composition detector within a wave number range of 4000-400 cm⁻¹, and outputting contents of ordinary milk ingredients and a transmittance of each of the collected milk samples by a computer connected to the milk composition detector;

3) data preprocessing, including:

converting original spectral data of the mid-infrared spectra from transmittance to absorbance, and removing outliers;

4) dataset dividing, including:

randomly dividing the dataset of the A2 milk samples and the non-A2 milk samples into a training set and a testing set respectively accounted for 80% and 20% of the dataset;

5) determining modeling spectral wavebands, including:

removing an absorption region of water, and screening difference wavebands between the non-A2 milk samples and the A2 milk samples;

6) model establishment, including:

taking the mid-infrared spectra of milk samples in the training set as input and classes of non-A2 milk and A2 milk as output, and establishing a classification model by using combinations of a random forest algorithm and different spectrum preprocessing methods; and

7) screening cows with A2 β-casein genotype of producing A2 milk, including:

substituting detected mid-infrared spectral data of milk into the established classification model for identification of A2 milk and non-A2 milk, identifying and screening the cow with a classification result of A2 milk as a A2A2 type cow with A2 β-casein genotype of producing A2 milk, otherwise identifying and screening the cow as non-A2A2 type cow.

According to the above-described identification and screening method, preferably, the step 3) includes: converting the transmittance T to the absorbance A as per a formula A=log₁₀(1/T), and reserving data of milk samples with normal contents of ordinary milk ingredients. The data of milk samples with normal contents of ordinary milk ingredients refer to data meeting conditions of a milk fat percentage in a range of 2-9 g/100 g, a milk protein percentage in a range of 1-7 g/100 g, a somatic cell count equal to or less than 1 million cells/mL, and a Mahalanobis distance equal to or less than 3; and a calculation method of the Mahalanobis distance is expressed as MD=sqrt[(x−μ)^{T}Σ⁻¹(x−μ)], where x represents a spectral value, μ represents a sample mean value, Σ represents a covariance matrix, and T represents transposition.

According to the above-described identification and screening method, preferably, in the step 5), the modeling spectral wavebands include: 925.92-1076.382 cm⁻¹, 1134.252-1257.708 cm⁻¹, 1473.756-1639.65 cm⁻¹, 1736.1-2465.262 cm⁻¹, and 2847.204-2970.66 cm⁻¹; and the step 6) includes: using first-order differentiation (Diff, its parameter is 1), SNV, MSC, and SG convolution smoothing with a window size being a combination of 17 in length and 3 in width individually to preprocess mid-infrared spectral data and compare with the mid-infrared spectral data without the preprocess (NONE), taking accuracy, sensitivity, specificity, and area under curve (AUC) as evaluation indexes, and determining to select the first-order differentiation and the random forest (RF) algorithm to establish a model A for identification of cows with A2 β-casein genotype. In the classification model established by using the random forest (RF) algorithm, a classification result with more than half of decision trees is used as a final result; when samples to be tested are divided into two classes, variable matrices in Y matrix (i.e., an output matrix) are replaced by ‘0’ and ‘1’ to mark different classes; and when a number of decision trees with the decision of ‘0’ is more than a number of decision trees with the decision of ‘1’, the sample to be tested is determined as ‘0’ (i.e., non-A2), namely, β-casein genotype of a cow corresponding to the sample to be tested is determined as non-A2A2 type, otherwise the sample to be tested is determined as ‘1’, namely, the β-casein genotype of the cow is determined as A2A2 type.

According to the above-described identification and screening method, preferably, in the step 5), the modeling spectral wavebands include: 925.92-1076.382 cm⁻¹, 1138.11-1269.282 cm⁻¹, 1323.294-1377.306 cm⁻¹, 1439.034-1469.898 cm⁻¹, 1504.62-1539.342 cm⁻¹, 1766.964-2835.63 cm⁻¹, and 2854.92-2966.802 cm⁻¹; and the step 6) includes: using first-order differentiation with a parameter of 1, SNV, MSC, and SG convolution smoothing with a window size being a combination of 17 in length and 3 in width individually to preprocess mid-infrared spectral data and compare with the mid-infrared spectral data without the preprocess, taking accuracy, sensitivity, specificity, and AUC as evaluation indexes, and determining to select none preprocessing of mid-infrared spectral data and the random forest (RF) algorithm to establish a model B for identification of cows with A2 β-casein genotype. In the classification model established by using the random forest (RF) algorithm, a classification result with more than half of decision trees is used as a final result; when samples to be tested are divided into two classes, variable matrices in Y matrix are replaced by ‘0’ and ‘1’ to mark different classes; and when a number of decision trees with the decision of ‘0’ is more than a number of decision trees with the decision of ‘1’, the sample to be tested is determined as ‘0’, namely, β-casein genotype of a cow corresponding to the sample to be tested is determined as non-A2A2 type, otherwise the sample to be tested is determined as ‘1’, namely, the β-casein genotype of the cow is determined as A2A2 type.

In another aspect, an embodiment of the invention provides an application of the model established by any one of the identification and screening methods in identifying cows with A2 β-casein genotype of producing A2 milk.

In still another aspect, an embodiment of the invention provides an application of the model A and the model B established by the above-described identification and screening methods in identifying cows with A2 β-casein genotype of producing A2 milk, including: judging a genotype of each of cows as one of A2A2 type and non-A2A2 type by the two models, individually; screening ones of the cows each with classification results of the two models both being a cow of A2A2 genotype; and determining the ones of the cows as A2A2 type cows with A2 β-casein genotype of producing A2 milk.

Accuracies of the model A and the model B for predicting A2 type cows may be 95.17% and 96.81% respectively, and thus it can achieve advantages of accurate, batch, automatic, fast and non-invasive.

According to the above-described application, preferably, when detecting, mid-infrared spectral data from milk samples of cows to be tested are acquired, and the acquired spectral data are substituted into the model A and the model B, and corresponding result files with the code “0” and “1” are output. A sample whose results of the two models are “0” indicates that the β-casein genotype of the cow is non-A2A2 type, and the sample whose results of the two models are “1” indicates that the β-casein genotype of the cow is A2A2 type.

Beneficial effects can be achieved by the embodiments of the invention may be as follows.

The fast, batch and non-invasive identification and screening method of cows with A2 β-casein genotype being A2A2 type of producing A2 milk can realize rapid and batch identification of cows of producing A2 milk and non A2 milk, and may have advantages of fast, high precision, low cost, simple operation, batch determination, no trauma (no need of blood collection, hair plucking, and tissue extraction), and strong practicability.

The identification and screening method of cows with A2 β-casein genotype being A2A2 type of producing A2 milk may have short determination time, high determination accuracy and high detection efficiency.

The method is the first in the world to use mid-infrared spectroscopy to identify different genotypes of dairy protein genes, and can reduce the cost compared with a molecular biology genotype detection technology.

The identification and screening method of cows with A2 β-casein genotype being A2A2 type of producing A2 milk may have the following advantages:

(1) the accuracy of the model may be up to 95% or more, and thus it can realize accurate screening and identification;

(2) the prediction time of the model may be only within 0.5 minutes per sample, and thus it can realize quick screening and identification;

(3) can carry out batch sample detection on a whole dairy herd, and thus it can realize batch low-consumption screening and identification;

(4) there is no need of carrying out chemical treatment on the milk samples, no need of chemical and molecular biological reagents, and thus it is beneficial to environmental protection and can realize green detection, screening and identification; and

(5) milk samples are used instead of collecting tissues, blood or hair (with hair follicles) of cow, and thus it can ensure a high-level of animal welfare and achieve non-invasive screening and identification of cows.

### BRIEF DESCRIPTION OF THE DRAWINGS

FIG. 1 illustrates a schematic spectrum of a model A according to embodiment 1.

FIG. 2 illustrates a schematic ROC curve of a testing set of the model A according to the embodiment 1.

FIG. 3 illustrates a schematic spectrum of a model B according to embodiment 2.

FIG. 4 illustrates a schematic ROC curve of a testing set of the model B according to the embodiment 2.

### DETAILED DESCRIPTION OF EMBODIMENTS

The following embodiments are intended to further illustrate the invention, but should not be construed as limiting the invention. Any modification or substitution made to the invention without departing from the spirit and essence of the invention belongs to the scope of the invention.

Unless otherwise specified, technical means used in the following embodiments are conventional means well known to those skilled in the art, and parameter adjustments of techniques such as first-order differential (Diff), standard normal variate transformation (SNV), multivariate scatter correction (MSC) and Savitzky-Golay (SG) convolution smoothing are conventional adjustments made by those skilled in the art according to research objects. Reagents or materials as used, unless otherwise specified, come from commercial sources.

In the following embodiments of the invention, mid-infrared spectral data preprocessing, and model construction and verification were all realized through Python 3.8.3. Detection of ordinary milk ingredients and a light transmittance of each sample was performed through a milk composition detector MilkoScantm7RM (operated as per its product instructions manual) produced by the FOSS company.

### Embodiment 1

Establishment of identification model A of cows with A2 β-casein genotype of producing A2 milk, may include specific steps as follows.

(1) Collecting Milk Samples

Raw milk uniformly mixed in a whole milking process were collected from a dairy farm of breeding non-A2 cows and A2 cows. In particular, 492 pieces of A2 milk were collected from 492 numbers of cows with β-casein genotype being A2A2 (shorted as A2), and 866 pieces of non-A2 milk were collected from 866 numbers of cows with non-A2A2 genotype (shorted as non-A2).

(2) Acquiring Mid-Infrared Spectra

Each of the collected milk samples was poured into a cylindrical sampling tube with a diameter of 3.5 cm and a height of 9 cm, a liquid level was ensured to be greater than 6 cm, then was bathed in a 42° C. water bath for 15-20 min, afterwards a solid optical fiber probe was extended into the liquid for sample aspirating detection, and a milk composition detector was used to scan the milk sample within a wave number range of 4000-400 cm⁻¹, and contents of ordinary milk ingredients and a light transmittance corresponding to each the milk sample were output through a connected computer. The ordinary milk ingredients include milk fat, milk protein, lactose, milk solids non-fat, total solids, urea, and somatic cell count.

(3) Data Preprocessing

For original spectral data, the light transmittances were converted to absorbances, and abnormal values (outliers) were removed. Specifically, the light transmittances (T) were converted to the absorbances (A) according to A=log₁₀(1/T); the abnormal values were removed by using the milk fat percentage, the somatic cell count and the Mahalanobis distance; and the data with the milk fat percentage in the range of 2-9 g/100 g, the milk protein percentage in the range of 1-7 g/100 g, the somatic cell count equal to or less than 1 million cells/mL ( [symbol] 1 million cells/mL), and the Mahalanobis distance of spectrum equal to or less than 3 ( [symbol] 3) were reserved. A calculation method of the Mahalanobis distance is MD=sqrt[(x−μ)^{T}Σ⁻¹(x−μ)], where x represents a spectral value, μ represents a sample mean value, Σ represents a covariance matrix, and T represents transposition. After removing the abnormal samples, there were 814 pieces of non-A2 milk data and 454 pieces of A2 milk data.

(4) Dataset Dividing

362 pieces of non-A2 milk data and 362 pieces of A2 milk data obtained in the step (3) were randomly selected as a modeling dataset which was randomly divided into a training set and a testing set, which accounted for 80% and 20% of the dataset respectively. The remaining 452 pieces of non-A2 milk data and 92 pieces of A2 milk data were used as a validation set.

(5) Determining Modeling Spectral Wavebands

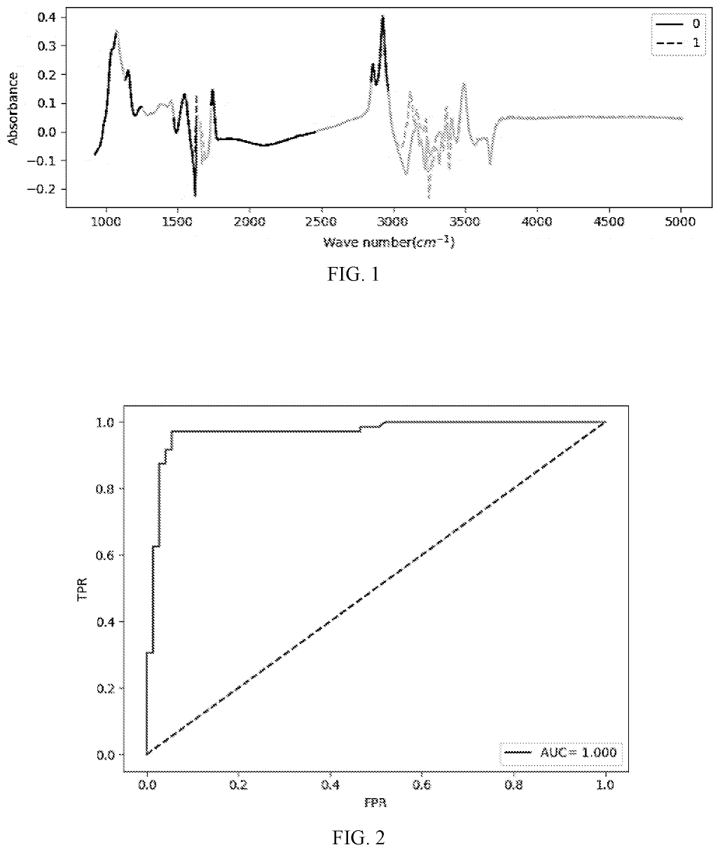

Spectral data of the modeling dataset were performed with Pearson correlation test, correlations were analyzed for significance, and an absorption region of water was removed. FIG. 1 shows an average spectrum of the modeling wavebands. In FIG. 1, the abscissa is the spectral wave number, and the ordinate is the absorbance. In the legend, “0” represents non-A2 milk, “1” represents A2 milk; The parameter involved is significant level a=0.05, and wavebands of P<0.05 were selected for modeling. Finally, 925.92-1076.382 cm⁻¹, 1134.252-1257.708 cm⁻¹, 1473.756-1639.65 cm⁻¹, 1736.1-2465.262 cm⁻¹ and 2847.204-2970.66 cm⁻¹ were selected for modeling.

(6) Model Establishment

The wavebands selected in the step (5) were used, spectral data are preprocessed by first-order differentiation (Diff, its parameter is 1), standard normal variable transformation (SNV), multivariate scatter correction (MSC) and SG convolution smoothing (a window size of the SG convolution smoothing is set to one of combinations of lengths 17, 19, 21, 23, 25, 27, 29, 31, 33 and widths 2, 3, 4, the combination with the best effect as per model evaluation indexes is selected as a modeling result of the SG convolution smoothing, and the window size selected in the illustrated embodiment of the invention is the combination of 17 in length and 3 in width) individually, and then compared with the spectral data of none preprocessing.

A random forest (RF) algorithm was used to establish classification models using data of the training set and to predict samples in the testing set.

Modeling results of the RF algorithm under different preprocessings are shown in Table 1 below.

TABLE 1
Modeling results of RF algorithm under different preprocessings

| | | | Evaluation indexes | | | |
|---|---|---|---|---|---|---|
| Algo- | Pre- | | Accu- | Sensi- | Speci- | |
| rithm | processing | Dataset | racy | tivity | ficity | AUC |
| RF | None | Training set | 1 | 1 | 1 | 1 |
| | | Testing set | 0.83 | 0.86 | 0.79 | 0.91 |
| | Diff | Training set | 1 | 1 | 1 | 1 |
| | | Testing set | 0.95 | 0.96 | 0.95 | 0.97 |
| | SNV | Training set | 1 | 1 | 1 | 1 |
| | | Testing set | 0.86 | 0.90 | 0.82 | 0.90 |
| | MSC | Training set | 1 | 1 | 1 | 1 |
| | | Testing set | 0.80 | 0.83 | 0.77 | 0.85 |
| | SG (17,3) | Training set | 1 | 1 | 1 | 1 |
| | | Testing set | 0.95 | 0.96 | 0.95 | 0.97 |

(7) Screening the Best Classification Model

In the above dichotomous models, the accuracy, the sensitivity, the specificity and the AUC (area under curve) are used to evaluate the performances of the models. The accuracy denotes a probability that correct judgments accounts for all judgments, and the better the closer the value thereof is to 1. The sensitivity denotes a proportion of a class correctly classified in the dichotomous model, and the better the closer the value thereof is to 1. The specificity denotes a probability of another class correctly classified in the dichotomous model, and the better the closer the valve thereof is to 1. AUC denotes an area under ROC (abbreviation of receiver operating characteristic) curve, which intuitively reflects a classification ability expressed by the ROC curve, AUC=1 means this classifier is a perfect classifier, 0.5<AUC<1 means this classifier is better than a random classifier, and 0<AUC<0.5 means this classifier is worse than the random classifier. As seen from Table 1, the models established by Diff and SG preprocessings have achieved better results in the classification training of A2 milk and non-A2 milk, and the two models can accurately identify two types of targets in the training set and the testing set. Since the Diff has a faster rate in the operation than the SG convolution smoothing, that is, the Diff preprocessed model is more efficient in this classification, and thus the Diff preprocessed RF model is selected as the best classification model (also referred to as optimal classification model).

The selected optimal classification model was used to predict 145 numbers of samples of the testing set, and results are shown in Table 2.

TABLE 2
Testing set results of the model

| | Number of | Number of correctly | | Total |
|---|---|---|---|---|
| Class | true samples | identified samples | Accuracy | accuracy |
| Non-A2 | 73 | 69 | 94.52% | 95.17% |
| A2 | 72 | 69 | 95.83% | |

A ROC curve is used to measure the performance of the model in the testing set. The abscissa FPR is the false positive rate, that is, the number of real non-A2 milk judged as A2 milk divided by the number of all real non-A2 milk in the testing set, and the ordinate TPR is the true positive rate, that is, the number of real A2 milk judged as A2 milk divided by the number of all real A2 milk in the testing set, and the ROC curve shown in FIG. 2 is obtained. AUC is an area enclosed by the coordinate axes under the ROC curve, and its value is between 0.5 and 1. The closer the AUC is to 1.0, the higher the authenticity of the embodiment of the invention. It can be seen from FIG. 2 that the AUC of the testing set in this embodiment is 1, indicating that the classification effect of the model on the testing set is very good.

In the random forest (RF) classification model, a classification result with more than half of decision trees was used as a final result; when samples were divided into two classes, variable matrices in the Y matrix were replaced by ‘0’ and ‘1’ to mark the different classes, and when the number of decision trees with the decision of ‘0’ was more than the number of decision trees with the decision of ‘1’, the sample is determined as ‘0’, namely, the β-casein genotype of the cow corresponding to the sample to be tested is non-A2A2 type, and otherwise the sample is determined as ‘1’, i.e., the β-casein genotype of the cow is A2A2 type.

(8) Validation of Optimal Model

The validation set was used to externally validate the model, and predicted results were compared with actual results.

Method: spectral data of samples in the validation set were substituted into the trained model, prediction results can be output, and external validation results are shown in Table 3.

TABLE 3
validation results of model

| | Number of | Number of correctly | | Total |
|---|---|---|---|---|
| Class | true samples | identified samples | Accuracy | accuracy |
| Non-A2 | 452 | 377 | 83.41% | 85.11% |
| A2 | 92 | 86 | 93.48% | |

It can be seen from the above that, the optimal model A with the highest prediction accuracy for identification of cows with A2 β-casein genotype was finally established as that: using normal milk samples (data with the milk fat percentage in the range of 2-9 g/100 g, the milk protein percentage in the range of 1-7 g/100 g, the somatic cell count equal to or less than 1 million cells/mL ( [symbol] 1 million cells/mL), and the Mahalanobis distance of spectrum equal to or less than 3 ( [symbol] 3) were reserved), five optimal modeling wavebands (925.92-1076.382 cm⁻¹, 1134.252-1257.708 cm⁻¹, 1473.756-1639.65 cm⁻¹, 1736.1-2465.262 cm⁻¹ and 2847.204-2970.66 cm⁻¹), the first-order differential (Diff, 1) mid-infrared spectral data preprocessing method, and the random forest (RF) algorithm to establish an optimal model for identification of cows with A2 β-casein genotype. The model may achieve a prediction accuracy for A2A2 type cows being 95%, which may have advantages of accurate, batch, automatic, fast and non-invasive. The accuracy of the validation set of the model was 85.11% (463/544), and prediction accuracies of cows of producing non-A2 milk and A2 milk were 83.41% and 93.48% respectively.

### Embodiment 2

Establishment of identification model B of cows with A2 β-casein genotype of producing A2 milk, may include specific steps as follows.

(1) Collecting Milk Samples

318 pieces of A2 milk from 318 numbers of A2 cows and 539 pieces of non-A2 milk from 539 numbers of non-A2 cows were further collected from the sampled dairy farm of the embodiment 1.

(2) Acquiring Mid-Infrared Spectra

Each of the collected milk samples was poured into a cylindrical sampling tube with a diameter of 3.5 cm and a height of 9 cm, a liquid level was ensured to be greater than 6 cm, then was bathed in a 42° C. water bath for 15-20 min, afterwards a solid optical fiber probe was extended into the liquid for sample aspirating detection, and a milk composition detector was used to scan the milk sample within a wave number range of 4000-400 cm⁻¹, and contents of ordinary milk ingredients and a light transmittance corresponding to each the sample were output through a connected computer.

(3) Data Preprocessing

For original spectral data, the light transmittances were converted to absorbances, abnormal values (outliers) were removed. Specifically, the light transmittances (T) were converted to the absorbances (A) according to A=log₁₀(1/T); the abnormal values were removed by using a milk fat percentage, a milk protein percentage, a somatic cell count and a Mahalanobis distance; and normal milk samples were used, data with the milk fat percentage in the range of 2-9 g/100 g, the milk protein percentage in the range of 1-7 g/100 g, the somatic cell count equal to or less than 1 million cells/mL ( [symbol] 1 million cells/mL), and the Mahalanobis distance equal to or less than 3 ( [symbol] 3) were reserved. A calculation method of the Mahalanobis distance is MD=sqrt[(x−μ)^{T}Σ⁻¹(x−μ)], where x represents a spectral value, μ represents a sample mean value, Σ represents a covariance matrix, and T represents transposition. After removing the abnormal samples, there were 510 pieces of non-A2 milk data and 299 pieces of A2 milk data.

(4) Dataset Dividing

260 pieces of non-A2 milk data and 260 pieces of A2 milk data were randomly selected as a modeling dataset which was randomly divided into a training set and a testing set, which accounted for 80% and 20% of the dataset respectively. The remaining 279 pieces of non-A2 milk data and 39 pieces of A2 milk data were used as a validation set.

(5) Determining Modeling Spectral Wavebands

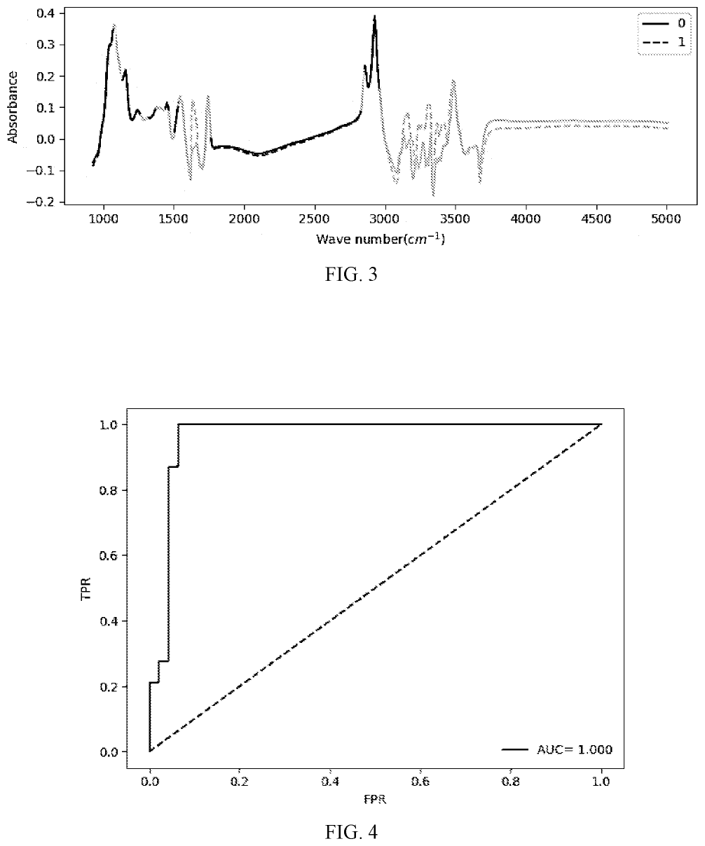

Spectral data were performed with Pearson correlation test, correlations were analyzed for significance, and an absorption region of water was removed. The parameter involved is significant level a=0.05, and wavebands of P<0.05 were selected for modeling. Finally, 925.92-1076.382 cm⁻¹, 1138.11-1269.282 cm⁻¹, 1323.294-1377.306 cm⁻¹, 1439.034-1469.898 cm⁻¹, 1504.62-1539.342 cm⁻¹, 1766.964-2835.63 cm⁻¹ and 2854.92-2966.802 cm⁻¹ were selected for modeling. FIG. 3 shows an average spectrum of the modeling wavebands.

(6) Model Establishment

Spectral data are preprocessed by first-order differentiation (Diff, its parameter is 1), standard normal variable transformation (SNV), multivariate scatter correction (MSC) and SG convolution smoothing (a window size of the SG convolution smoothing is set to one of combinations of lengths 17, 19, 21, 23, 25, 27, 29, 31, 33 and widths 2, 3, 4, the combination with the best effect as per model evaluation indexes is selected as a modeling result of the SG convolution smoothing, and the window size selected in the illustrated embodiment of the invention is 17 in length and 3 in width) individually, and then compared with the spectral data of none preprocessing.

A random forest (RF) algorithm was used to establish classification models using data of the training set and to predict samples in the testing set.

Modeling results of the RF algorithm under different preprocessings are shown in Table 4 below.

TABLE 4
Modeling results of RF algorithm under different preprocessings

| | | | Evaluation indexes | | | |
|---|---|---|---|---|---|---|
| Algo- | Pre- | | Accu- | Sensi- | Speci- | |
| rithm | processing | Dataset | racy | tivity | ficity | AUC |
| RF | None | Training set | 1 | 1 | 1 | 1 |
| | | Testing set | 0.97 | 1 | 0.94 | 0.97 |
| | Diff | Training set | 1 | 1 | 1 | 1 |
| | | Testing set | 0.94 | 0.98 | 0.89 | 0.96 |
| | SNV | Training set | 1 | 1 | 1 | 1 |
| | | Testing set | 0.80 | 0.82 | 0.77 | 0.84 |
| | MSC | Training set | 1 | 1 | 1 | 1 |
| | | Testing set | 0.71 | 0.77 | 0.66 | 0.81 |
| | SG (17,3) | Training set | 1 | 1 | 1 | 1 |
| | | Testing set | 0.95 | 0.98 | 0.91 | 0.96 |

(7) Screening the Best Classification Model

As seen from Table 4, the RF model established based on the none preprocessing achieved a better result in the classification training of A2 milk and non-A2 milk, the model can accurately identify two types of targets in the training set and the testing set, and thus the RF model without preprocessing was selected as the best classification model (also referred to as optimal classification model).

In the random forest (RF) established classification model, a classification result with more than half of decision trees was used as a final result; when the samples were divided into two classes, variable matrices in the Y matrix were replaced by ‘0’ and ‘1’ to mark the different classes, and when the number of decision trees with the decision of ‘0’ was more than the number of decision trees with the decision of ‘1’, the sample is determined as ‘0’, i.e., the β-casein genotype of the cow corresponding to the sample to be tested is non-A2A2 type, and otherwise the sample is determined as ‘1’, i.e., the β-casein genotype of the cow is A2A2 type.

The selected optimal classification model was used to predict 94 numbers of samples of the testing set, and results are shown in Table 5.

TABLE 5
Testing set results for the model

| | Number of | Number of correctly | | Total |
|---|---|---|---|---|
| Class | true samples | identified samples | Accuracy | accuracy |
| Non-A2 | 47 | 44 | 93.62% | 96.81% |
| A2 | 47 | 47 | 100% | |

A ROC curve is used to measure the performance of the model in the testing set. the false positive rate (FPR) was used as the abscissa, the true positive rate (TPR) was used as the ordinate TPR, and the ROC curve shown in FIG. 4 was obtained. It can be seen from FIG. 4 that the AUC of the testing set in this embodiment is 1, indicating that the classification effect of the model on the testing set is very good. Therefore, none preprocessing was selected as the data preprocessing method for modeling.

(8) Validation of Optimal Model

The validation set was used to externally validate the model, and predicted results were compared with real results.

Method: spectral data of samples in the validation set were substituted into the trained model, prediction results can be output, and external validation results are shown in Table 6.

TABLE 6
validation results of model

| | Number of | Number of correctly | | Total |
|---|---|---|---|---|
| Class | true samples | identified samples | Accuracy | accuracy |
| Non-A2 | 279 | 252 | 90.32% | 91.19% |
| A2 | 39 | 38 | 97.44% | |

It can be seen from the above that, the optimal model B with the highest prediction accuracy for identification of cows with A2 β-casein genotype was finally established as that: using normal milk samples (data with the milk fat percentage in the range of 2-9 g/100 g, the milk protein percentage in the range of 1-7 g/100 g, the somatic cell count equal to or less than 1 million cells/mL ( [symbol] 1 million cells/mL), and the Mahalanobis distance equal to or less than 3 ( [symbol] 3) were reserved), seven optimal modeling wavebands (925.92-1076.382 cm⁻¹, 1138.11-1269.282 cm⁻¹, 1323.294-1377.306 cm⁻¹, 1439.034-1469.898 cm⁻¹, 1504.62-1539.342 cm⁻¹, 1766.964-2835.63 cm⁻¹ and 2854.92-2966.802 cm⁻¹), the mid-infrared spectral data being not preprocessed (NONE), and the random forest (RF) algorithm to establish an optimal model for identification of cows with A2 β-casein genotype. The model may achieve a prediction accuracy for A2 type cows being 97%. The accuracy of the validation set of the model was 91.19% (290/318), and prediction accuracies of cows producing non-A2 milk and A2 milk were 90.32% and 97.44% respectively, and the model may have advantages of accurate, batch, automatic, fast and non-invasive.

### Embodiment 3

The identification model A and the identification model B were used simultaneously to screen cows with A2 β-casein genotype of producing A2 milk.

The model A and the model B were used to perform prediction on 481 numbers of cows (including 163 numbers of cows with true A2A2 genotype and 318 numbers of cows with non-A2A2 genotype), and specific methods and steps may be as follows.

(1) mid-infrared spectral data of milk samples of the 481 numbers of cows were acquired.

(2) the acquired spectral data were input into the model A and the model B individually, and corresponding result files each with “0” or “1” as the code were output.

(3) a sample with results of the two models both being “0” denotes the β-casein genotype of the cow is non-A2A2 type, and a sample with results of the two models both being “1” denotes the β-casein genotype of the cow is A2A2 type.

(4) cows determined as being with A2A2 type β-casein genes by both the two models were selected, and the accuracy of the selected cows was 100%, and the results are shown in Table 7.

TABLE 7
prediction results of model A and model B

| | | Inconsistent | True | | | Non-A2 | A2 | Accuracy of | Frequency of |
|---|---|---|---|---|---|---|---|---|---|
| | Screened | total number | A2 | Screened | Screened | misjudged | misjudged | screened A2 | occurrence of |
| Model | cows | of two models | cow | A2 cow | true A2 | as A2 | as non-A2 | cows | A2 cows |
| A | 481 | | 163 | 165 | 160 | 5 | 3 | 98.16%(160/163) | 33.26% |
| | | | | | | | | | (160/481) |
| B | 481 | | 163 | 181 | 157 | 24 | 6 | 96.32%(157/163) | 33.26% |
| | | | | | | | | | (157/481) |
| A + B | 481 | 36 | 154 | 155 | 154 | 1 | 0 | 100%(154/154) | 32.02% |
| | | | | | | | | | (154/481) |

The model A was used to predict and preliminarily screen 165 numbers of cows with A2A2 genotype. In which, 160 (true) numbers of cows with A2A2 genotype were predicted from the 163 numbers of cows with true A2A2 genotype, 5 numbers of cows with non-A2A2 genotype were misjudged as cows with A2A2 genotype, and 3 numbers of cows with A2A2 genotype were misjudged as cows with non-A2A2 genotype, so that the accuracy of predicting the cows with A2A2 genotype was 98.16% (160/163), and a screening rate of predicting the cows with A2A2 genotype was 33.26% (160/481).

The model B was used to predict and preliminarily screen 181 numbers of cows with A2A2 genotype. In which, 157 (true) numbers of cows with A2A2 genotype were predicted from the 163 numbers of cows with true A2A2 genotype, 24 numbers of cows with non-A2A2 genotype were misjudged as cows with A2A2 genotype, and 6 numbers of cows with A2A2 genotype were misjudged as cows with non-A2A2 genotype, so that the accuracy of predicting the cows with A2A2 genotype was 96.32% (157/163), and a screening rate of predicting the cows with A2A2 genotype was 32.64% (157/481).

The model A and the model B were simultaneously used for screening, except for 36 numbers of cows with inconsistent results of the two models, 155 numbers of cows with A2A2 genotype were screened from 291 numbers of non-A2A2 cows and 154 numbers of A2A2 cows. In which, 154 (true) numbers of cows with A2A2 genotype were predicted from the 154 numbers of cows with true A2A2 genotype, one cow with non-A2A2 genotype was misjudged as the cow with A2A2 genotype, and zero cow with A2A2 genotype was misjudged as the cow with non-A2A2 genotype, so that the accuracy of predicting the cows with A2A2 genotype was 100% (154/154), and a screening rate of predicting the cows with A2A2 genotype was 32.02% (154/481). Through the combination of the two models, although the frequency of screening A2 cows was reduced by 0.24% (only 3-6 numbers of cows were reduced), the accuracy and reliability of screening A2 were increased by 2-3%, which could reach the accuracy of 100%, and the result is shown in above Table 7.

Therefore, during determining results and selecting A2A2 type cows, one with identical results of the model A and the model B is used as an accurate result. In particular, when both the models predict a sample to be A2 milk, the sample is determined as A2 milk and the cow producing the milk sample is determined as a cow with A2 β-casein genotype of producing A2 milk. For milk samples with inconsistent results predicted by the two models, the samples should be removed or not selected. By applying the prediction model(s), especially the simultaneous use of the two models and the selection of cows whose predicted results of the two models both are A2 milk producers, which not only can greatly save the cost of detection (compared with genetic detection), with only 0.5 min per sample to obtain the detection result, but also can have advantages of fast, high precision, simple operation, batch determination, non-invasive (no need of blood collection, hair plucking, tissue extraction) and high-level of animal welfare, thereby presenting strong practicality and applicability.

What is claimed is:

1. An identification and screening method of cows with A2 β-casein genotype of producing A2 milk, comprising following steps:
(1) collecting milk samples, comprising:
collecting raw milk from a dairy farm of breeding cows with A2 β-casein genotype of producing A2 milk and cows with non-A2 genotype of producing non-A2 milk, to obtain A2 β-casein milk (A2 milk) samples and non-A2 β-casein milk (non-A2 milk) samples;
(2) acquiring mid-infrared spectra, comprising:
scanning collected milk samples by using a milk composition detector within a wave number range of 4000-400 cm⁻¹, and outputting contents of ordinary milk ingredients and a transmittance of each of the collected milk samples by a computer connected to the milk composition detector;
(3) data preprocessing, comprising:
converting original spectral data of the mid-infrared spectra from transmittance to absorbance, and removing outliers;
(4) dataset dividing, comprising:
randomly dividing a modeling dataset of the A2 milk samples and the non-A2 milk samples obtained after the step (3) into a training set and a testing set respectively accounted for 80% and 20% of the modeling dataset;
(5) determining modeling spectral wavebands, comprising:
removing an absorption region of water, and screening difference wavebands between the non-A2 milk samples and the A2 milk samples;
(6) model establishment, comprising:
taking the mid-infrared spectra of milk samples in the training set as input and classes of non-A2 milk and A2 milk as output, and establishing a classification model by using combinations of a random forest algorithm and different spectrum preprocessing methods; and
(7) screening cows with A2 β-casein genotype of producing A2 milk by using the established classification model, comprising:
substituting detected mid-infrared spectral data of milk into the established classification model for identification of A2 milk and non-A2 milk, identifying and screening the cow with a classification result of A2 milk as a A2A2 type cow with A2 β-casein genotype of producing A2 milk, otherwise identifying and screening the cow as non-A2A2 type cow.

2. The identification and screening method according to claim 1, wherein the step (3) comprises: converting the transmittance T to the absorbance A as per a formula A=log 10(1/T), and reserving data of milk samples with normal contents of ordinary milk ingredients;
wherein the data of milk samples with normal contents of ordinary milk ingredients refer to data meeting conditions of a milk fat percentage in a range of 2-9 g/100 g, a milk protein percentage in a range of 1-7 g/100 g, a somatic cell count equal to or less than 1 million cells/mL, and a Mahalanobis distance equal to or less than 3; and a calculation method of the Mahalanobis distance is expressed as MD=sqrt[(x−μ)^{T}Σ⁻¹(x−μ)], where x represents a spectral value, μ represents a sample mean value, Σ represents a covariance matrix, and T represents transposition.

3. The identification and screening method according to claim 1, wherein in the step (5), the modeling spectral wavebands comprise: 925.92-1076.382 cm⁻¹, 1134.252-1257.708 cm⁻¹, 1473.756-1639.65 cm⁻¹, 1736.1-2465.262 cm⁻¹, and 2847.204-2970.66 cm⁻¹;
wherein the step (6) comprises: using first-order differentiation with a parameter of 1, standard normal variable transformation (SNV), multivariate scatter correction (MSC), and Savitzky-Golay (SG) convolution smoothing with a window size being a combination of 17 in length and 3 in width individually to preprocess mid-infrared spectral data and compare with the mid-infrared spectral data without the preprocess, taking accuracy, sensitivity, specificity, and area under curve (AUC) as evaluation indexes, and determining to select the first-order differentiation and the random forest algorithm to establish a model A for identification of cows with A2 β-casein genotype.

4. The identification and screening method according to claim 1, wherein in the step (5), the modeling spectral wavebands comprise: 925.92-1076.382 cm⁻¹, 1138.11-1269.282 cm⁻¹, 1323.294-1377.306 cm⁻¹, 1439.034-1469.898 cm⁻¹, 1504.62-1539.342 cm⁻¹, 1766.964-2835.63 cm⁻¹, and 2854.92-2966.802 cm⁻¹;
wherein the step (6) comprises: using first-order differentiation with a parameter of 1, SNV, MSC, and SG convolution smoothing with a window size being a combination of 17 in length and 3 in width individually to preprocess mid-infrared spectral data and compare with the mid-infrared spectral data without the preprocess, taking accuracy, sensitivity, specificity, and AUC as evaluation indexes, and determining to select none preprocessing of mid-infrared spectral data and the random forest algorithm to establish a model B for identification of cows with A2 β-casein genotype.

5. The identification and screening method according to claim 1, wherein in the step (7), in the classification model established by using the random forest algorithm, a classification result with more than half of decision trees is used as a final result; when samples to be tested are divided into two classes, variable matrices in an output matrix are replaced by ‘0’ and ‘1’ to mark different classes; and when a number of decision trees with the decision of ‘0’ is more than a number of decision trees with the decision of ‘1’, the sample to be tested is determined as ‘0’ and thus β-casein genotype of a cow corresponding to the sample to be tested is determined as non-A2A2 type, otherwise the sample to be tested is determined as ‘1’ and thus the β-casein genotype of the cow is determined as A2A2 type.

6. A use of the model established by the identification and screening method according to claim 1 in identifying cows with A2 β-casein genotype of producing A2 milk.

7. A use of two models respectively being the model A established by the identification and screening method according to claim 4 and the model B established by the identification and screening method according to claim 5, comprising:
judging a genotype of each of cows as one of A2A2 type and non-A2A2 type by the two models, individually;
screening ones of the cows each with classification results of the two models both being a cow of A2A2 genotype; and
determining the ones of the cows as A2A2 type cows with A2 β-casein genotype of producing A2 milk.
